(19) United States
(12) Patent Application Publication
(10) Pub. No.: US 2024/0325794 A1
(43) Pub. Date: Oct. 3, 2024

(54) SYSTEMS AND METHODS FOR EXTRACELLULAR VESICLE RELEASE

(71) Applicant: THE TRUSTEES OF COLUMBIA UNIVERSITY IN THE CITY OF NEW YORK, New York, NY (US)

(72) Inventors: Elisa E. Konofagou, New York, NY (US); Alec J. Batts, New York, NY (US); Alina R. Kline-Schoder, New York, NY (US)

(73) Assignee: THE TRUSTEES OF COLUMBIA UNIVERSITY IN THE CITY OF NEW YORK, New York, NY (US)

(21) Appl. No.: 18/625,639

(22) Filed: Apr. 3, 2024

Related U.S. Application Data
(60) Provisional application No. 63/456,641, filed on Apr. 3, 2023.

(51) Int. Cl. A61N 7/02; A61N 7/00

(52) U.S. Cl. CPC A61N 7/02; A61N 2007/0021; A61N 2007/0039

(57) ABSTRACT

The present subject matter relates to techniques for releasing extracellular vesicles. The disclosed system can include an ultrasound assembly configured to apply an ultrasound treatment to a target tissue of the subject, a sampling device configured to collect a sample from the subject, and an analysis device configured to analyze a biomarker in the sample. The ultrasound assembly can include an ultrasound transducer and a function generator.

### CROSS-REFERENCE TO RELATED APPLICATION

This application claims priority to U.S. Provisional Patent Application Nos. 63/456,641, which was filed on Apr. 3, 2023, the entire contents of which are incorporated by reference herein.

### GRANT INFORMATION

This invention was made with government support under EB009041, and AG038961 awarded by the National Institutes of Health. The government has certain rights in the invention.

### BACKGROUND

The blood-brain barrier (BBB) is a barrier between the blood and the brain that keeps the brain at homeostasis for neuronal firing. While the BBB limits the infiltration of neurotoxins and pathogens, it also limits both the delivery of drugs to the brain and the circulation of neurological disease biomarkers in the blood. Focused-ultrasound blood-brain barrier opening (FUS-BBBO) combines focused ultrasound with intravenously administered microbubbles to transiently and noninvasively open the BBB to overcome such challenges.

Extracellular vesicles (EVs) are lipid vesicles responsible for cell transport and exchange. EVs have variable cargo, including proteins, carbohydrates, and/or coding and non-coding RNA (ncRNA). Due to their small size, there can be an emphasis on ncRNA within EVs. Certain documented types of ncRNA are micro RNA (miRNA) and piwi-interacting RNA (piRNA). Due to their role in intercellular communication, EV isolation can be a method to improve the specificity of biomarker detection.

Therefore, there is a need for improved systems and methods for treating patients with neuro disorders with EV and FUS-BBBO.

### SUMMARY

The disclosed subject matter provides techniques for releasing extracellular vesicles (EV).

An example system can include an ultrasound assembly configured to apply an ultrasound treatment to a target tissue of the subject, a sampling device configured to collect a sample from the subject, and an analysis device configured to analyze a biomarker in the sample.

In certain embodiments, the ultrasound transducer can include a spherical-segment focused ultrasound transducer, a pulse-echo ultrasound transducer, a linear array transducer, or combinations thereof. In non-limiting embodiments, the ultrasound assembly can be configured to apply a focused ultrasound (FUS) treatment, a theranostic-ultrasound (ThUS) treatment, or a combination thereof.

In certain embodiments, the ultrasound treatment has one or more ultrasound parameters. In non-limiting embodiments, the ultrasound parameters can include a center frequency, a focal depth, a focal area, one of a peak negative pressure, a stimulation duration, a duty cycle, a pulse repetition frequency (PRF), or combinations thereof.

In certain embodiments, the transducer can be configured to apply the ultrasound treatment to a blood-brain barrier. In non-limiting embodiments, the sample can include blood, serum, or a combination thereof. In non-limiting embodiments, the sample can be collected about 1 hour after the ultrasound treatment.

In certain embodiments, the biomarker can include an inflammation-related marker, a proliferation-associated gene, an immediate inflammatory response-related protein, a hemoglobin-associated protein, or combinations thereof. In non-limiting embodiments, the biomarker can include alpha-synuclein (aSyn), amyloid-beta, tau, or combinations thereof.

In certain embodiments, the ultrasound transducer can be configured to apply the FUS treatment at about 0.25 MHz center frequency or the ThUS treatment at about 0.5 MHz center frequency.

An example method for releasing extracellular vesicles (EVs) of a subject can include applying an ultrasound treatment to a target tissue of the subject, collecting a sample from the subject, isolating EVs from the sample, and analyzing a biomarker in the sample.

In certain embodiments, ultrasound treatment can include a focused ultrasound (FUS) treatment, a theranostic-ultrasound (ThUS) treatment, or a combination thereof. In non-limiting embodiments, the method can further include adjusting a parameter of the ultrasound treatment. In non-limiting embodiments, the parameter can include a center frequency, a focal depth, a focal area, one of a peak negative pressure, a stimulation duration, a duty cycle, a pulse repetition frequency (PRF), or combinations thereof.

In certain embodiments, the biomarker can include alpha-synuclein (aSyn), amyloid-beta, tau, or combinations thereof. In non-limiting embodiments, the biomarker can include an inflammation-related marker, a proliferation-associated gene, an immediate inflammatory response-related protein, a hemoglobin-associated protein, or combinations thereof.

In certain embodiments, the sample can include blood, serum, or a combination thereof. In non-limiting embodiments, the target tissue can include a brain or a blood-brain barrier. In non-limiting embodiments, the sample can be collected about 1 hour after the ultrasound treatment.

In certain embodiments, the subject can have a neurological disorder. In non-limiting embodiments, the method can further include applying an effective amount of therapeutic treatment to a subject based on the biomarker.

The disclosed subject matter will be further described below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D provide diagrams and graphs showing that extracellular vesicle concentration increases after the treatment of focused-ultrasound blood-brain barrier opening (FUS-BBBO) in accordance with the disclosed subject matter.

FIGS. 2A-2F provide diagrams and graphs showing that FUS-BBBO can alter extracellular vesicle RNA and protein load in accordance with the disclosed subject matter.

FIGS. 3A-3F provide diagrams and graphs showing that GW4869 can reduce murine EV concentration increase and inflammatory response in accordance with the disclosed subject matter.

FIGS. 4A-4E provide diagrams and graphs showing that extracellular vesicle concentration increases after FUS-BBBO in Alzheimer's patients in accordance with the disclosed subject matter.

FIGS. 5A-5D provide diagrams and graphs showing that patient extracellular vesicles can alter content after FUS-BBBO in accordance with the disclosed subject matter.

FIG. 6 provides a graph showing the concentration of EVs in the serum of mice following THUS-BBBO in accordance with the disclosed subject matter.

FIG. 7 provides a graph showing the concentration of EVs in the serum of NHPs following blood-brain barrier opening with and without AAV in accordance with the disclosed subject matter.

FIGS. 8A-8B provide graphs showing the log fold change in concentration of ΔD biomarkers in serum and in EVs 3 days after FUS-BBBO (FIG. 8A). The change of some biomarkers correlates with the BBB opening volume (FIG. 8B) in accordance with the disclosed subject matter.

It is to be understood that both the foregoing general description and the following detailed description are exemplary and are intended to provide further explanation of the disclosed subject matter.

### DETAILED DESCRIPTION

The disclosed subject matter provides techniques for releasing extracellular vesicles (EVs). The disclosed subject matter provides systems and methods for releasing extracellular vesicles with an ultrasound treatment.

The terms “comprise(s),” “include(s),” “having,” “has,” “can,” “contain(s),” and variants thereof, as used herein, are intended to be open-ended transitional phrases, terms, or words that do not preclude additional acts or structures. The singular forms “a,” “an,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and up to 1% of a given value. Alternatively, e.g., with respect to biological systems or processes, the term can mean within an order of magnitude, within 5-fold, and within 2-fold of a value.

As used herein, an “effective amount” refers to an amount of the compound sufficient to treat, prevent, manage the disease or to generate a physiological response. An effective amount can refer to the amount of a compound that provides a beneficial physiological response in the treatment or management of the disease, and as such, an “effective amount” depends upon the context in which it is being applied. In the context of administering anesthetics during FUS modulation in a subject, an effective amount of anesthetics described herein is an amount sufficient to elicit an anesthetizing effect in the subject. An effective amount can be administered in one or more administrations. Further, a therapeutically effective amount can mean the amount of therapeutic alone, or in combination with other therapies, that provides a therapeutic benefit in the treatment or management of the disease, which can include a decrease in the severity of disease symptoms, an increase in frequency and duration of disease symptom-free periods, or a prevention of impairment or disability due to the disease affliction. The term can encompass an amount that improves overall therapy, reduces or avoids unwanted effects, or enhances the therapeutic efficacy of or synergies with another therapeutic agent.

As used herein, the term “subject” includes any human or non-human animal. The term “non-human animal” includes, but is not limited to, all vertebrates, e.g., mammals and non-mammals, such as non-human primates, dogs, cats, sheep, horses, cows, chickens, amphibians, reptiles, etc.

As used herein, “treatment” or “treating” refers to inhibiting the progression of a disease or disorder or delaying the onset of a disease or disorder, whether physically, e.g., stabilization of a discernible symptom, physiologically, e.g., stabilization of a physical parameter, or both. As used herein, the terms “treatment,” “treating,” and the like, refer to obtaining a desired pharmacologic and/or physiologic effect. The effect can be prophylactic in terms of completely or partially preventing a disease or condition, or a symptom thereof and/or can be therapeutic in terms of a partial or complete cure for a disease or disorder and/or adverse effect attributable to the disease or disorder. “Treatment,” as used herein, covers any treatment of a disease or disorder in an animal or mammal, such as a human, and includes: decreasing the risk of death due to the disease; preventing the disease or disorder from occurring in a subject which can be predisposed to the disease but has not yet been diagnosed as having it; inhibiting the disease or disorder, i.e., arresting its development (e.g., reducing the rate of disease progression); and relieving the disease, i.e., causing regression of the disease.

An example system can include an ultrasound assembly, a sampling device, and an analysis device. The ultrasound assembly can be configured to apply an ultrasound treatment to a target tissue of the subject. For example, the ultrasound assembly can include an ultrasound transducer and a function generator. In non-limiting embodiments, the ultrasound assembly can be configured to apply a focused ultrasound (FUS) treatment, a theranostic-ultrasound (ThUS) treatment, or a combination thereof. In certain embodiments, the FUS treatments refer to the use of a geometrically focused single-element transducer for the induction of BBB opening and a secondary transducer, concentrically and coaxially aligned with the focused transducer for cavitation monitoring. In non-limiting embodiments, the ThUS treatments refer to the use of a repurposed imaging phased array to combine electronically focused pulses to induce BBB opening with cavitation monitoring in a single transducer.

In certain embodiments, the transducer can include a spherical-segment focused ultrasound transducer, a pulse-echo ultrasound transducer, a linear array transducer, or combinations thereof. In non-limiting embodiments, the transducer can be configured to deliver an ultrasound treatment with a predetermined ultrasound parameter. For example, the ultrasound parameters can include a center frequency, a focal depth, a focal area, one of a peak negative pressure, a stimulation duration, a duty cycle, a pulse repetition frequency (PRF), or combinations thereof.

In certain embodiments, the center frequency can range from about 20 kilohertz (kHz) to about 10 megahertz (MHz). In non-limiting embodiments, the center frequency can range from about 0.1 MHz to about 10 MHz, from about 0.1 MHz to about 5 MHZ, from about 0.1 MHz to about 1.5 MHz, from about 0.1 MHz to about 1 MHz, from about 0.1 MHz to about 0.5 MHz, or from about 0.1 MHz to about 0.25 MHz. In non-limiting embodiments, the center frequency of the FUS stimulation probe can be about 0.25 or 1.5 MHz. In non-limiting embodiments, the center frequency of the ThUS stimulation probe can range from about 0.2 MHZ to about 1.5 MHz.

In certain embodiments, the focal depth can range from about 1 mm to about 1 cm, from about 1 mm to about 500 mm, from about 1 mm to about 100 mm, from about 1 mm to about 60 mm, from about 1 mm to about 50 mm, from about 1 mm to about 40 mm, from about 1 mm to about 30 mm, from about 1 mm to about 20 mm, or from about 1 mm to about 10 mm. In non-limiting embodiments, In non-limiting embodiments, the focal depth is about 60 mm.

In certain embodiments, the predetermined ultrasound parameter can include the diameter and radius curvature of the disclosed element transducer. The diameter of the single-element transducer can range from about 10 millimeters (mm) to about 200 mm, from about 30 mm to about 150 mm, from about 30 mm to about 110 mm, from about 40 mm to about 110 mm, from about 50 mm to about 110 mm, or from about 60 mm to about 110 mm. In some embodiments, the radius of curvature can range from about 30 millimeters (mm) to about 200 mm, from about 30 mm to about 150 mm, from about 30 mm to about 110 mm, from about 40 mm to about 110 mm, from about 50 mm to about 110 mm, from about 60 mm to about 110 mm, or from about 70 mm to about 110 mm.

In certain embodiments, the predetermined ultrasound parameter can include a pulse length, pulse repetition frequency, peak-negative pressure, and sonication duration. For example, the pulse length can range from about 0.001 milliseconds (ms) to about 100 ms, from about 0.001 ms to about 90 ms, from 0.001 ms to about 80 ms, from 0.001 ms to about 70 ms, from 0.001 ms to about 60 ms, from 0.001 ms to about 50 ms, from 0.001 ms to about 40 ms, from 0.001 ms to about 30 ms, from 0.001 ms to about 20 ms, or from 0.001 ms to about 10 ms. The pulse length can also range from about 1 cycle to about 5000 cycles, from about 1 cycle to about 4000 cycles, from about 1 cycle to about 10,000 cycles, from about 1 cycle to about 5000 cycles, from about 1 cycle to about 4000 cycles, from about 1 cycle to about 3000 cycles, from about 1 cycle to about 2500 cycles, from about 500 cycles to about 2500 cycles, from about 1000 cycles to about 2500 cycles, from about 1500 cycles to about 2500 cycles, or from about 2000 cycles to about 2500 cycles. The pulse repetition frequency can range from about 0.1 Hz to about 10 kHz, from about 0.1 Hz to about 9 kHz, from about 0.1 Hz to about 8 kHz, from about 0.1 Hz to about 7 kHz, from about 0.1 Hz to about 6 kHz, from about 0.1 Hz to about 5 kHz, from about 0.1 Hz to about 4 kHz, from about 0.1 Hz to about 3 kHz, or from about 0.1 Hz to about 2 kHz.

In certain embodiments, the sonication duration can range from about 1 second to about 5 minutes, from about 0.1 minutes to about 4 minutes, from about 0.1 minutes to about 3 minutes, from about 0.1 minutes to about 2 minutes, from about 0.5 minutes to about 2 minutes, or from about 1 minute to about 2 minutes. In non-limiting embodiments, the sonication duration can be about 2 minutes. In non-limiting embodiments, the target tissue can be sonicated more than one time.

In certain embodiments, the peak-negative pressure can range from about 0.1 MPa to about 10 MPa, from about 0.1 MPa to about 9 MPa, from about 0.1 MPa to about 8 MPa, from about 0.1 MPa to about 7 MPa, from about 0.1 MPa to about 6 MPa, from about 0.1 MPa to about 5 MPa, from about 0.1 MPa to about 4 MPa, from about 0.1 MPa to about 3 MPa, from about 0.1 MPa to about 2 MPa, from about 0.1 MPa to about 1 MPa, from about 0.1 MPa to about 0.5 MPa, from about 0.1 MPa to about 0.4 MPa, from about 0.1 MPa to about 0.3 MPa, or from about 0.1 MPa to about 0.2 MPa. In non-limiting embodiments, the peak-negative pressure can be about 0.2 MPa.

In certain embodiments, the disclosed system can include microbubbles. The microbubbles can be configured to react to a predetermined pulse of the disclosed ultrasound treatment and induce cavitation for opening the target tissue. The size of the microbubbles can range from about 1 micron to about 10 microns, from about 1 micron to about 9 microns, from about 1 micron to about 8 microns, from about 1 micron to about 7 microns, from about 1 micron to about 6 microns, from about 1 micron to about 6 microns, from about 1 micron to about 5 microns, from about 2 microns to about 5 microns, from about 3 microns to about 5 microns, or from about 4 microns to about 5 microns. In non-limiting embodiments, the dose of the microbubbles can be adjusted depending on the subject. For example, clinical doses (e.g., about 10 μl/kg) of microbubbles for ultrasound imaging applications can be administered to a human subject.

In certain embodiments, the disclosed system can induce the cavitation of microbubbles to open the target tissue by applying the disclosed ultrasound treatment. For example, the disclosed FUS or ThUS treatment can induce the cavitation of microbubbles to open the blood-brain barrier of a subject.

In certain embodiments, the disclosed system can include a sampling device configured to collect a sample from the subject. The sample device can include a device configured to collect blood or serum from the subject. In non-limiting embodiments, the sampling device can include a serum collection tube, a blood collection tube (e.g., a whole-blood collection tube), or a combination thereof. For example, blood can be collected from veins and can be left at room temperature for about 15-30 minutes to clot and then spun down at 3000×g for 15 minutes to obtain serum. Serum can be aliquoted and stored at −80 C. In non-limiting embodiments, EVs can be isolated from the collected serum.

In certain embodiments, the disclosed system can include an analysis device configured to analyze a biomarker in the sample. The analysis device can be configured to perform EV concentration measurements, protein quantification, RNA sequencing, immunoassay, PCR, or combinations thereof. For example, the concentration and size distribution of EVs can be evaluated using Nanoparticle Tracking Analysis (NTA). The genomic and proteomic content of EVs can be evaluated using RNA sequencing, multiplex assay, western blotting, and/or mass spectrometry.

In certain embodiments, the biomarker can include biomarkers of Parkinson's disease (PD) and/or Alzheimer's disease (AD). For example, the analysis device can analyze alpha-synuclein (aSyn), amyloid-beta, tau, or combinations thereof. In non-limiting embodiments, the biomarker can include an inflammation-related marker, a proliferation-associated gene, an immediate inflammatory response-related protein, a hemoglobin-associated protein, or combinations thereof. For example, inflammation-related markers (e.g., Mapk12), proliferation-associated genes (e.g., Sox3), immediate inflammatory response-related proteins (e.g., Lpb), and/or hemoglobin-associated proteins (e.g., Hbb-bs) can be analyzed by the disclosed analysis device.

The disclosed subject matter provides methods for releasing EVs. An example method can include applying an ultrasound treatment to a target tissue of the subject, collecting a sample from the subject, isolating EVs from the sample, and analyzing a biomarker in the sample. In non-limiting embodiments, the ultrasound treatment can be a focused ultrasound (FUS) treatment, a theranostic-ultrasound (ThUS) treatment, or a combination thereof.

In certain embodiments, the target tissue can be any tissue. For example, the target tissue can be a nerve, a brain, a heart, muscle, tendons, ligaments, skin, vessels, blood-brain barrier, a subcortical brain structure, a hippocampus, a caudate-putamen, a brain parenchyma, or a combination thereof.

In certain embodiments, the sample can be collected from the subject after a predetermined time. For example, the predetermined time can be about 5 minutes, about 10 minutes, about 15 minutes, about 20 minutes, about 30 minutes, about 45 minutes, about 1 hour, about 2 hours, about 3 hours, about 4 hours, about 5 hours, or about 10 hours after the ultrasound treatment. In non-limiting embodiments, the sample can be collected about 1 hour after the ultrasound treatment. In non-limiting embodiments, the sample can include blood, serum, or a combination thereof.

In certain embodiments, the subject can include a patient with neurodegenerative diseases or neural disorders (e.g., Alzheimer's, Parkinson's, and amyotrophic lateral sclerosis).

In certain embodiments, EVs can be isolated from the collected samples. For example, blood can be collected from a vein and left at room temperature to clot. Serum can be separated and diluted with PBS. The serum can be incubated with an EV separation agent (e.g., Exoquick) at room temperature. The samples can then be spun down, and the pellet (isolated extracellular vesicles) can be resuspended in 1×PBS.

In certain embodiments, various biomarkers can be analyzed. For example, the biomarker can include biomarkers of Parkinson's disease (PD) and/or Alzheimer's disease (AD). For example, the analysis device can analyze alpha-synuclein (aSyn), amyloid-beta, tau, or combinations thereof. In non-limiting embodiments, the biomarker can include an inflammation-related marker, a proliferation-associated gene, an immediate inflammatory response-related protein, a hemoglobin-associated protein, or combinations thereof. For example, inflammation-related markers (e.g., Mapk12), proliferation-associated genes (e.g., Sox3), immediate inflammatory response-related proteins (e.g., Lpb), and/or hemoglobin-associated proteins (e.g., Hbb-bs) can be analyzed by the disclosed analysis device.

In certain embodiments, the method can further include applying an effective amount of a therapeutic treatment to a subject based on the biomarker. In non-limiting embodiments, the therapeutic treatment can include a pharmacological agent administration, an antibody administration, an administration of a viral or non-viral vector for gene delivery to a target region, or combinations thereof.

In certain embodiments, the method can further include adjusting a parameter of the ultrasound treatment. The parameter can include a center frequency, a focal depth, a focal area, one of a peak negative pressure, a stimulation duration, a duty cycle, a pulse repetition frequency (PRF), or combinations thereof. For example, FUS-BBBO can be conducted using the H-231 transducer operating at about 0.25 MHz. ThUS-BBBO can be carried out by a customized linear array operating at about 0.5 MHz.

### EXAMPLES

### Example 1: Focused-ultrasound blood-brain barrier opening alters murine and patient serum extracellular vesicle (EV) concentration and content

The disclosed subject matter provides systems and methods for treating neuro disorders. In this example, the concentration and content of isolated EVs from the serum of mice and Alzheimer's Disease (AD) patients before and after drug-free FUS-BBBO were identified. An increase in the murine EV concentration of over 100% 1 hour after treatment. To further elucidate the functional role of EVs within the neuroimmune response to FUS-BBBO, the EV depletion drug GW4869 was used, finding an elimination of the FUS-BBBO-induced EV concentration can increase as well as a reduction of the inflammatory response to FUS-BBBO. AD patient EV concentration increases by 100% 1 hour after treatment and remains dependent on the volume of blood-brain barrier opening at 3 days after treatment. 3 days after treatment, EV isolation improves the detection of AD biomarkers and other markers of neurological origin in proportion to the volume of the blood-brain barrier opening.

FUS-BBBO Treatment: all mice (FUS-BBBO and Sham) were anesthetized with oxygen and 1-2% isoflurane, and placed on a stereotaxis with head immobilized and depilated to reduce acoustic impedance mismatch. The suture was identified to position the transducer. In mice that were treated with FUS-BBBO, a single-element, spherical-segment FUS transducer (e.g., center frequency: 1.5 MHz, focal depth: 60 mm, radius: 30 mm) that was driven by a function generator through a 50-dB power amplifier was used to treat the bilateral hippocampus. The center of the transducer held a pulse-echo ultrasound transducer (e.g., center frequency: 10 MHz, focal depth: 60 mm, radius 11.2 mm) that was used for alignment and obtainment of cavitation data. The pulse-echo ultrasound transducer was driven by a pulse-receiver connected to a digitizer. The transducer setup was attached to a three-dimensional positioning system. Each hippocampus was sonicated first for 10 s for cavitation baseline and then again for 2 m for the experimental sonication. Microbubbles were injected intravenously between sonication. In all mice treated with MB (FUS-BBBO and Sham), a bolus of 3 μL of lab-made microbubbles (8×10e8/mL, mean diameter: 1.4 μm) was diluted with 100 μL sterile saline and then injected intravenously. The transducer was not triggered to treat MB Sham mice; otherwise, the treatment was identical to that of the FUS-BBBO mice.

Following treatment, all FUS-BBBO, MB Sham, and FUS Sham animals underwent scanning with a 9.4T MRI system. Mice were intraperitoneally injected with 0.2 mL of gadodiamide 30 min prior to scanning. Images were acquired using a contrast-enhanced T1-weighted 2D FLASH sequence (TR/TE 230/3.3 ms, flip angle: 70°, number of excitations: 6, field of view: 25.6 mm×25.6 mm).

Mouse Serum Collection: mouse blood was collected from the mouse submandibular vein without anesthesia. Mice were held by grasping the skin behind the head firmly. A 16 g was inserted at the submandibular vein and then removed. Less than 100 uL of blood was collected in heparin-coated serum-separating tubes. After collection, gentle pressure was applied to the site of the puncture in order to stop bleeding. After collection, blood was left at room temperature for 15-30 min to clot and then spun down at 3000×g for 15 min. The serum was aliquoted and stored at −80 C for future processing.

Extracellular Vesicle Isolation: 25 uL of mouse serum was diluted for the mouse samples with 75 uL of 1×PBS before incubation with 22.5 uL of Exoquick for 30 minutes at room temperature. The samples were then spun down, and the pellet (isolated extracellular vesicles) was resuspended in 1×PBS and stored at −80 Celsius until further processing.

Nanosight Analysis: extracellular vesicle concentration analysis was performed on a Nanosight. 5 uL of 1×PBS re-suspended extracellular vesicles was diluted into 995 uL of 1×PBS. This solution was then run through the Nanosight at a rate of 1000 uL/min, and the resulting image was captured and analyzed for particle concentration and size distribution.

Multiplex Luminex Assay: a Luminex multiplex assay was used to quantify proteins in the clinical blood and isolated clinical extracellular vesicles (LuminexCorp). Single procartaplex kits were purchased and combined to make a custom multiplex panel for analysis (ThermoFisher). Data were fit with a separate five-parameter logistic dose-response curve for each protein, and all curves had R2 greater than or equal to 0.95.

Mass Spectrometry Protein Analysis: mass spectrometry proteomics was performed on already isolated EVs by Systems Biosciences. Systems Biosciences lysed the isolated EVs in a gel-loading buffer, followed by gel-based extraction and trypsinization for peptide library creation for LC/MS ESI-TOF. Peptide signatures were then mapped to a database of known protein sequences. Peptide quantification across all four runs was loaded into R, normalized, and processed for differential protein expression utilizing the UniprotR package. All functional annotation was performed with the well-know TopGO package.

RNA Sequencing: RNA Sequencing was performed on already isolated EVs by Systems Biosciences. EV RNA was isolated and quantified using Agilent Bioanalyzer Small RNA Assay before 75 bp single-end read Next Gen Sequencing libraries were prepared with Qiagen small RNA library preparation and gel purification. Sequencing was performed on Illumina NextSeq with SE75 at an approximate depth of 10-15 million reads per sample. Reads were processed and aligned to the GRCm38 genome with Ensemble transcriptome annotation (GRCm38.p6) using CellRanger with default parameters. Count tables were loaded into R and underwent normalization and differential gene expression analysis with the edgeR package. piRNA and miRNA targets were extracted from piRNAdb and miRBase, respectively. All functional annotation was performed with the TopGO package.

Western blotting: western blotting was performed using an automated capillary immunoassay method. Samples and reagents were loaded onto an assay plate and put into the western blotting machine. The sample was automatically loaded and separated by size while it traveled through the stacking and separation matrix. Then, the separated proteins are fixed with proprietary capture chemistry. Target proteins are identified with primary and secondary HRP-conjugated antibodies.

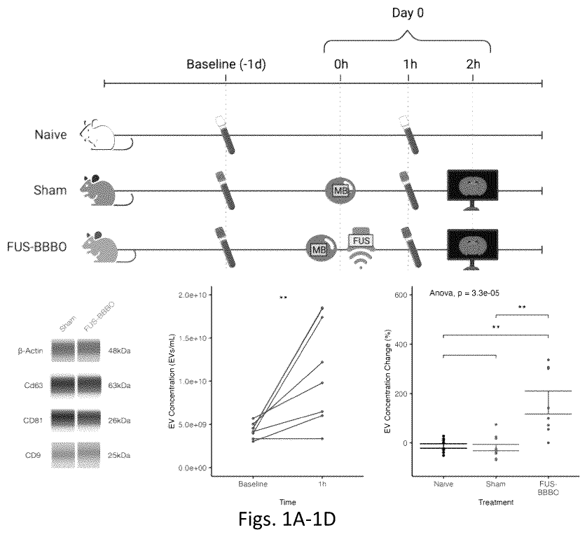

Murine extracellular vesicle concentration increases 1 hour after FUS-BBBO: wild-type mice were separated into three groups-Naive, Sham, and FUS-BBBO (FIG. 1A). Animals treated with FUS-BBBO were intravenously injected with microbubbles (MB) and treated with 2 minutes of focused ultrasound on the bilateral hippocampus. Animals in the Sham group were intravenously injected with MB but were not treated with focused ultrasound. Animals in all groups had blood drawn twice immediately before treatment (Baseline) and 1 hour after treatment (1 hour). Animals in the Sham and FUS-BBBO groups underwent contrast-enhanced T1-weighted MRI after the second blood draw to confirm the opening within the FUS-BBBO group and the lack of opening within the Sham group. The methods detail the extracellular vesicle (EV) isolation and concentration quantification.

Western blot of the isolated EVs from representative Sham and FUS-BBBO samples confirmed the successful isolation of the EVs via expression of marker proteins CD9, CD81, and CD63 (FIG. 1B). The EV concentration is significantly increased 1 hour after treatment compared to the Baseline (FIG. 1B). Furthermore, the average increase of 164% after FUS-BBBO is significantly higher than the percentage change in Naive and Sham samples, which averages nearly 0% (FIG. 1C).

FIGS. 1A-1D shows that mouse extracellular vesicle concentration increases after FUS-BBBO. As shown in FIG. 1A, blood draws were taken for extracellular vesicle isolation at two-time points-prior to treatment (Baseline) and 1 hour after treatment (1 hour). Animals were split into three treatment groups-FUS-BBBO, Sham, and Naive. Mice in the FUS-BBBO group had 2 min of FUS applied bilaterally following MB injection at time 0. Animals in the Sham group were injected with MB, but no FUS was applied. Mice from the FUS-BBBO and Sham group were injected with gadolinium (Gd), and a T1-Weighted MRI was performed less than 2 h following the initiation of the procedure. Animals in the Naive group were anesthesia-, MB-, and FUS-naive. FIG. 1B shows the western blot of B-actin and EV markers CD9, CD63, and CD81 on representative Sham and FUS-BBBO EV from 3 days post-treatment. FIG. 1C shows extracellular vesicle concentration (determined by Nanosight) at baseline and 3 days post-treatment in mouse serum. Paired T-test was performed and displayed on the graph. FIG. 1D shows extracellular vesicle concentration 3 days after treatment (determined by Nanosight) normalized to Baseline concentration for Naive, Sham, and FUS-BBBO groups. ANOVA followed by Bonferroni-corrected t-test between groups.

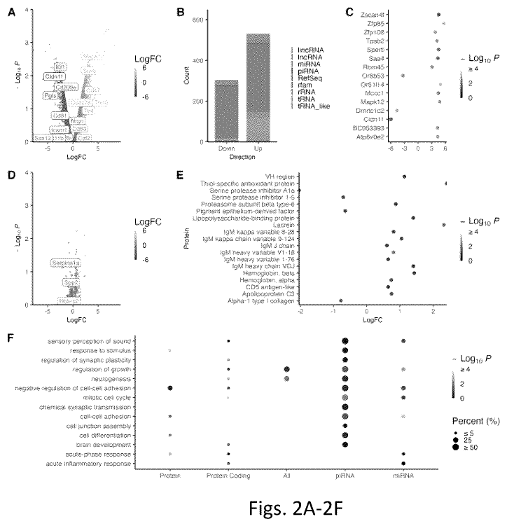

FUS-BBBO alters murine extracellular vesicle protein and RNA load: given the significant increase in EV concentration 1 hour after treatment, whole genome RNA-sequencing and mass spectrometry protein identification (Systems Biosciences) were performed on isolated EVs from Baseline and 1 hour. From each time point, two samples pooled from 3 animals underwent both processes. Differential gene expression analysis between 1 hour and baseline reveals significantly up- and down-regulated protein-coding and non-protein-coding (ncRNA) RNA (FIGS. 2A-2C). Upregulated protein-coding genes include proliferation-associated genes such as Sox3 and inflammation-associated genes such as Mapk12. Down-regulated protein-coding genes include tight-junction genes such as Cldn11. Differential expression of the protein identification finds many fewer significantly up and down-regulated proteins (only 10 proteins as compared to 900 genes). The most significantly upregulated proteins include immediate inflammatory response proteins such as Lbp and hemoglobin-associated proteins such as Hbb-bs (FIG. 2D-E).

Next, functional annotation was used to identify the biological processes associated with the differentially expressed: a) proteins, b) protein-encoding genes, c) ncRNA, d) piRNA target genes, and e) miRNA target genes. Protein functional annotation maps to immediate and acute inflammatory response compared to the protein-coding and non-coding RNA, which map to more long-term responses such as synapse regulation and neurogenesis (FIG. 2F). Many of the functions associated with the RNA changes are coincident with reported FUS-BBBO increases in neurogenesis, proliferation, and synaptic remodeling. This leads to the hypothesis of EV response involvement in the neuroimmunotherapeutic responses to FUS-BBBO.

FIGS. 2A-2F shows that FUS-BBBO alters murine extracellular vesicle RNA and protein load. FIG. 2A shows the schematic of RNA Sequencing and Mass Spectrometry Protein analysis. Isolated extracellular vesicles from baseline and 1-hour post-treatment from six mice were analyzed. Extracellular vesicles were pooled between three mice, and two runs were performed for both time points. FIG. 2B shows the Volcano plot of differentially expressed RNA between 1-hour post-treatment and Baseline extracellular vesicles with key protein-coding genes highlighted. FIG. 2C shows the plot of the most significantly up and down-regulated protein-coding RNA with Log FC of protein expression on the x-axis and significance shown in size. FIG. 2D shows bar charts of the significantly (>0.05) up and down-regulated RNA split by type. FIG. 2E shows the Volcano plot of differentially expressed proteins (from mass spectrometry proteomics) between 1-hour post-treatment and Baseline extracellular vesicles with key proteins highlighted. FIG. 2F shows plots of the most significantly up and down-regulated proteins with Log FC of protein expression on the x-axis and significance shown in size. FIG. 2G shows genes detected in both protein and RNA with Log FC from 1-hour post-treatment compared to baseline in the color and significance of the change expressed with stars on the heatmap. FIG. 2H shows the functional annotation of 1) upregulated proteins, 2) significantly upregulated protein-coding genes, 3) upregulated non-coding RNA (ncRNA), 4) genetic targets of the significantly upregulated piRNA, and 5) genetic targets of the significantly upregulated micro RNA (miRNA). Adjusted Kolmogorov-Smirnov p-value magnitude is displayed in color. The size of each dot corresponds to the percentage of annotated genes from that term that are significantly upregulated.

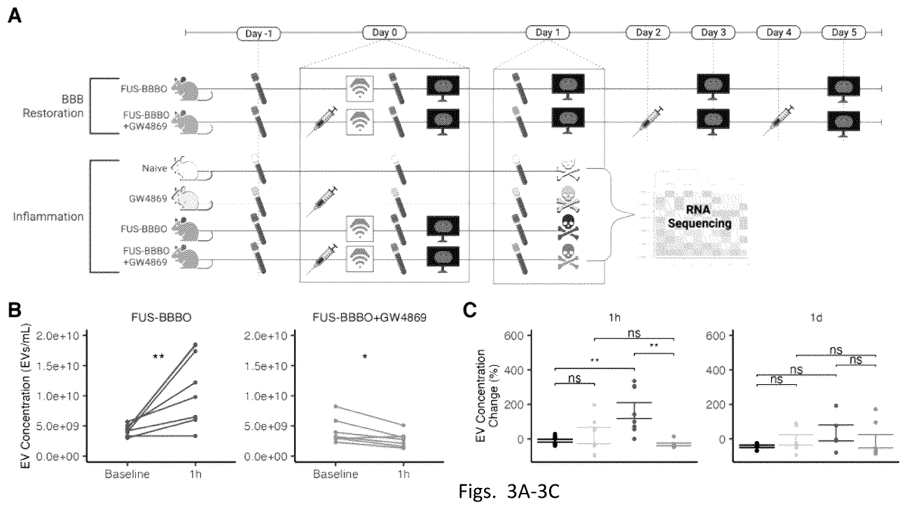

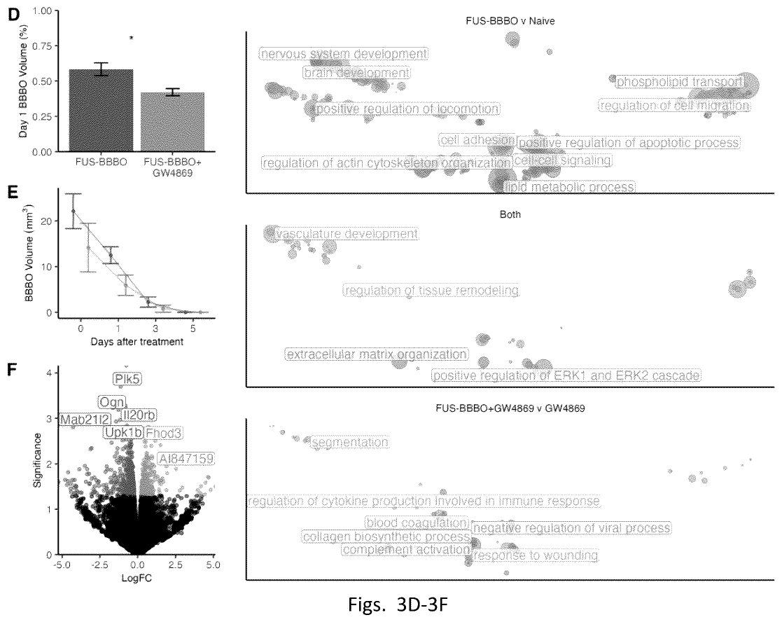

GW4869 eliminates murine EV concentration increase and reduces inflammatory response: to further elucidate the role of the EVs in modulating FUS-BBBO neuroimmunotherapy, GW4869, a neutral sphingomyelinase inhibitor that is the most widely used agent for blocking EV generation, was used (FIG. 3A). Four groups were assessed: Naive, Naive+GW4869, FUS-BBBO, and FUS-BBBO+GW4869. GW4869 successfully eliminated the FUS-BBBO-induced increase in EV concentration. One hour after treatment, the animals treated with FUS-BBBO+GW4869 had a statistically lower EV concentration compared to baseline, starkly contrasting with the FUS-BBBO group, which increased EV concentration by over 100% (FIG. 3B). Comparing the EV concentration change between four groups, FUS-BBBO+GW4869 is indistinct from Naive and Naive+GW4869 1 hour and 1 day after treatment (FIG. 3C).

Next, BBB restoration was monitored after FUS-BBBO with and without GW4869. The BBB opening volume of each animal was quantified on Days 0, 1, 3, and 5. On every day of measurement, the animals in the FUS-BBBO+GW4869 had smaller openings than those in the FUS-BBBO group. This difference is particularly dramatic and significant on day 1 (1 day) when the volumes of the FUS-BBBO group averaged 58% open compared with 42% open in the FUS-BBBO+GW4869 group (FIGS. 3D-3E).

FIGS. 3A-3F shows that GW4869 can eliminate murine EV concentration increase and reduce inflammatory response. FIG. 3A shows the GW4869 model timeline. In the BBB restoration assessment, sequential MRIs were taken after FUS-BBBO to monitor BBB restoration after treatment with and without GW4869. In the inflammation assessment, animals were sacrificed 1 day after treatment, the peak of the FUS-BBBO and inflammatory response and the bulk transcriptome was compared between FUS-BBBO, FUS-BBBO with GW4869, naive and naive+GW4869 animals. FIG. 3B shows extracellular vesicle concentration at baseline and 1 hour after treatment with either FUS-BBBO or FUS-BBBO+GW4869. A paired t-test was performed for each group. FIG. 3C shows the percent change in EV concentration 1 hour and 1 day after treatment for all groups. ANOVA followed by Bonferroni post hoc t-tests were performed for each time point. FIG. 3D shows the proportion of original blood-brain barrier opening volume that is still open 1 day after treatment for animals in the restoration study. An unpaired t-test was performed between the two groups. FIG. 3E shows the blood-brain barrier opening volume for the day of treatment and 1 day, 3 days, and 5 days following treatment for both FUS-BBBO and FUS-BBBO+GW4869. FIG. 3F shows the volcano plot comparing the profile of FUS-BBBO+GW4869 and FUS-BBBO hippocampi. Significant (pᵢ0.05) genes are colored. FIG. 3G shows significantly upregulated terms from differential gene expression analysis between FUS-BBBO+GW4869 and naive+GW4869 and FUS-BBBO compared with naive. Functional ontology terms are clustered by similarity, and the point size shows their significance. Terms appearing in both functional annotations are shown in the “Both” panel with the average significance between the two comparisons.

As 1 day was identified as the peak of FUS-BBBO-induced inflammation coupled with the more restored BBB in the FUS-BBBO+GW4869 group at this time point, bulk RNA sequencing was performed on tissue extracted from 1 day after FUS-BBBO for the four treatment groups. Differential gene expression between FUS-BBBO and FUS-BBBO+GW4869 revealed that GW4869 reduced the presence of inflammatory markers, including IL6 and CCL4 (FIG. 3F). In order to account for any effects of GW4869 treatment alone, functional annotation was performed on the differentially expressed genes between FUS-BBBO compared with Naive and FUS-BBBO+GW4869 compared with Naive+GW4869. This revealed increased processes altered in FUS-BBBO without injecting GW4869, including migratory, development, and inflammatory terms. The functions shared by both comparisons are primarily involved with vasculature development (FIG. 3G).

GW4869 eliminates the FUS-BBBO-induced increase in EV concentration, decreases the volume of BBB opening, and reduces the number of differentially affected processes after FUS-BBBO, indicating a vital role of EVs within the neuroimmunotherapeutic response to FUS-BBBO.

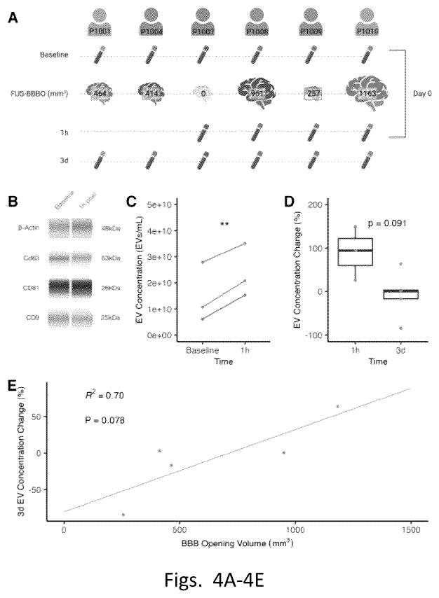

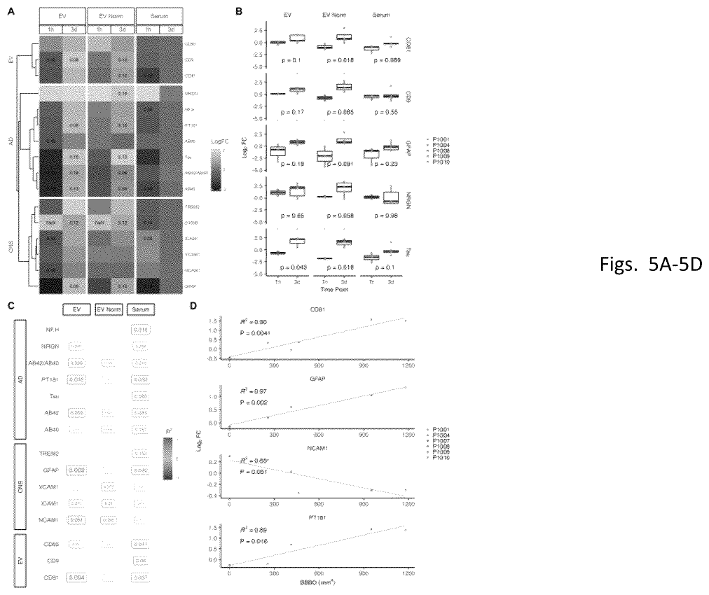

Patient extracellular vesicle concentration peaks 1 hour after FUS-BBBO: six Alzheimer's Clinical Patients underwent FUS-BBBO as part of the group's phase I clinical trial (NCT04118764). All patients had blood drawn immediately prior to treatment (Baseline) and 3 days after treatment. Additionally, the last four patients—P1007, P1008, P1009, and P1010 had blood drawn 1 hour after treatment (FIG. 4A).

EVs were isolated from each time point for each patient. Western blotting of the isolated EVs from representative Baseline and FUS-BBBO samples confirmed accurate EV isolation (FIG. 4B). Comparing EV concentration from baseline to 1 hour post-treatment reveals a significant increase in EV concentration (FIG. 4B) with a near-return to baseline by 3 days after treatment (FIG. 4C). Furthermore, the percent increase in EVs 3 days after treatment is correlated with the volume of the blood-brain barrier opening (FIG. 4D).

FIG. 4A-4D show that extracellular vesicle concentration increases after FUS-BBBO in Alzheimer's Patients. FIG. 4A shows the schematic of clinical treatment with FUS-BBBO, including each patient's blood draw and opening volume. FIG. 4B shows the percent change in EV concentration 1 hour and 3 days after treatment compared to baseline. An unpaired two-tail t-test was performed between the two groups. FIG. 4C shows the raw extracellular vesicle concentration at baseline and 1 hour after treatment for the patients who had successful treatment sessions. FIG. 4D shows the correlation of BBBO volume and the percent change in EV concentration 3 days after treatment. Simple linear regression was performed, and the resulting R2 and P-value are on the chart.

FUS-BBBO temporally alters patient extracellular vesicle protein load: to elucidate the utility of EVs in improving liquid biopsy specificity, several potential AD biomarkers, EV proteins, and other CNS proteins were quantified within the patient-isolated EVs, isolated EVs normalized by EV concentration (normalized EVs), and total serum. The EV, normalized EV, and total serum protein concentrations remained mostly unchanged 1 hour after treatment and even decreased compared to baseline. Three days after treatment, the concentration of a number of proteins is significantly increased for both EVs and normalized EVs compared to the total serum, which has no changes in marker concentration for any of the markers (FIG. 5A). Furthermore, the differences between 1 hour and 3 days Log FC in protein content are more statistically distinct for the EVs and normalized EVs than the total serum (FIG. 5B).

The MRI-based volume of the blood-brain barrier opening was compared to the Log FC of each biomarker 3 days after treatment. The level of the AD biomarkers is positively correlated with BBBO in serum and EV content but not normalized EV content. This indicates that the increase in detection is because more EVs are released, not because each EV has higher biomarker concentrations. Furthermore, compared to total serum, isolating the EVs proves to have a more significant correlation with BBBO for most of the AD biomarkers, including PT181 (p-value of 0.016 vs. 0.036) and AB42 (p-value of 0.058 vs. 0.093) (FIG. 5C-D).

FIGS. 5A-5D shows that the patient's extracellular vesicles alter content after FU-BBBO. FIG. 5A shows the log fold-change of raw and normalized EV content and serum content concentration 1 day and 3 days after treatment. The paired significance between the baseline and the concentration at each time is written on each cell. FIG. 5B shows the log fold-change (Log FC) of select markers 1 hour and 3 days after treatment for raw and normalized extracellular vesicle (EV) content and serum content. The significance of an unpaired t-test performed between the groups is displayed on each graph. FIG. 5C shows the correlation of blood-brain barrier opening (BBBO) volume and marker Log FC 1 hour and 3 days after treatment. Simple linear regression was performed for each condition. Color illustrates correlation strength with the significance of the correlation displayed numerically and in the size of the cell. FIG. 5D shows the correlation of blood-brain barrier opening (BBBO) volume and Log FC of EV marker concentration 3 days after treatment for select markers. Simple linear regression was performed for each condition, and R2 and P values are displayed on each graph.

A significant increase in mouse EV concentration was identified 1 hour after treatment coincident with RNA changes associated with the EV-dependent neuroimmunotherapeutic effects of FUS-BBBO, such as neurogenesis, synaptic pruning, and barrier maintenance. Eliminating the EV concentration increase with GW4869 resulted in reduced blood-brain barrier opening volume and inflammation, indicating the contribution of the EVs to FUS-BBBO inflammation and opening volume. The immune response to FUS-BBBO has spearheaded debate about the method's safety, so the ability to control and mitigate FUS-BBBO-induced inflammation provides a novel avenue, mainly when FUS-BBBO is used as a drug delivery tool.

A significant increase in Alzheimer's Disease (AD) patient EV concentration was identified 1 hour after treatment that remained dependent on BBBO volume 3 days after treatment. This was coupled with increased AD biomarker detection specificity in isolated EVs compared to total serum. However, in order to be implemented for improving biomarker specificity, there needs to be a method of determining what change in biomarker concentration is due to the treatment itself and what is due to the disease. Certain techniques (e.g., binary biomarkers such as cfDNA) can be utilized, present in the case of disease or otherwise completely absent. This becomes much more complex with diseases such as AD, where many of the proposed biomarkers are CNS proteins that are always in the CNS, albeit in different concentrations.

This example shows FUS-BBBO increasing EV concentration and altering EV content, which has implications in both the FUS-BBBO neuroimmunotherapy mechanism and the FUS-BBBO liquid biopsy optimization.

### Example 2: EV and Liquid Biopsy

Liquid biopsy refers to the detection of biomarkers in biological fluids without the need for invasive tissue collection and, therefore, presents an exciting alternative method for longitudinal monitoring of neurological disease treatments. In this example, EV quantification before and after FUS-BBBO and theranostic-ultrasound (ThUS)-mediated BBB opening (ThUS-BBBO) were assessed.

Exosomes in the blood of mice: for the isolation of EVs from the blood serum of mice that were treated with FUS-BBBO, a precipitation method was used with the commercially available ExoQuick (Systems Biosciences). Three groups of mice were used: a naïve group, which did not receive anesthesia or FUS-BBBO; a sham group, which received anesthesia but not FUS-BBBO; and a FUS-BBBO group that was sonicated unilaterally at the right hippocampus with a 450 kPa peak negative pressure. A single element, spherical segment FUS transducer was used and operated at 1.5 MHz. Blood was collected from the submandibular vein of mice before (baseline) and 1 hour after FUS-BBBO or sham treatment. Whole blood was centrifuged for 5 minutes at 9,400×g to separate serum from cells, and the serum was stored at −80 Celcius until all samples were collected. An equal amount of serum from all samples was aliquoted to sterile microtubes and centrifuged at 3000×g to separate cellular debris, and the supernatant containing EVs was aspirated and placed in another tube. Sterile 1×PBS was added to bring the solution's total volume to 100 μL, which was incubated with ExoQuick according to the manufacturer's instructions.

After incubation, two rounds of centrifugation were carried out to separate a pellet of EVs from other dissolved components of the serum, and the separated EVs were resuspended in sterile 1×PBS. The concentration and size distribution of EVs was evaluated using Nanoparticle Tracking Analysis (NTA) on a Nanosight NS300 (Malvern Panalytical).

Additionally, the genomic and proteomic content of EVs was evaluated using RNA sequencing and mass spectrometry. For RNA sequencing, the RNA was immediately isolated after euthanasia and sequenced on an Illumina NextSeq instrument. Libraries were prepared with Qiagen's small RNA library preparation kit. Processing of various miRNA targets was performed on R, with functional annotation being done using the TopGO package. For the mass spectrometry analysis, peptide libraries were created by Systems Biosciences and time-of-flight peptide signatures were loaded on a database of known protein sequences for analysis. Quantification and functional annotation were performed using R.

The NTA experiments revealed a significant increase in EV concentration in the serum of mice 1 hour after FUS-BBBO treatment. An average increase of 164% in EV concentration was measured for the FUS-BBBO mouse groups, compared to the sham and naïve groups, which averaged a near 0% change in EV concentration. Additionally, the genomic load of EVs post-FUS-BBBO was significantly changed, with an upregulation in inflammation-related markers such as Mapk12 and proliferation-associated genes such as Sox3. Finally proteomic analysis revealed an upregulation in immediate inflammatory response-related proteins such as Lpb and hemoglobin-associated proteins like Hbb-bs.

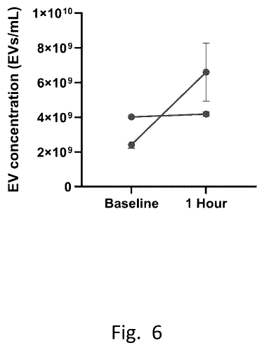

As shown in FIG. 6, the treatment with ThUS-BBBO and AAV delivery in two mice showed an increase in the concentration of EVs 1 hour post ThUS-BBBO. In the first mouse, the concentration of EVs increased by 174%, whereas in the second mouse, a smaller increase of 4% was observed. Accordingly, ThUS-BBBO can result in enhanced release of EVs in the blood circulation as soon as 1 hour after treatment.

Exosomes in the serum of NHPs: extracellular vesicles in serum collected from two rhesus macaques were assessed; one was subjected to FUS-BBBO treatment, and one received a combination of FUS-BBBO, ThUS-BBBO and AAV delivery. FUS-BBBO was conducted using the H-231 transducer (Sonic Concepts) operating at 0.25 MHz, and ThUS-BBBO was carried out by a customized linear array (L500, Vermon S.A.) operating at 0.5 MHz. The AAV construct delivered systemically in the second NHP was AAV9-CAG-GFP.

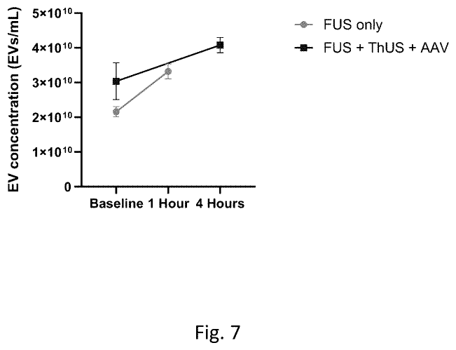

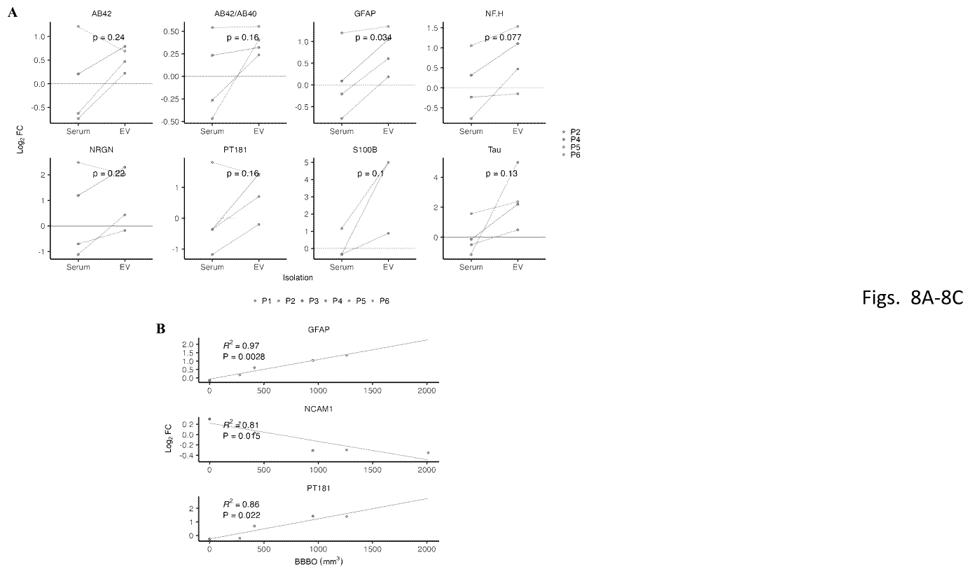

Blood samples were collected from the two non-human primates (NHP) at baseline and 1 to 4 hours after the sonication procedure. The serum was separated by centrifugation, and exosomes were isolated using the commercially available ExoQuick precipitation solution. Using Nanoparticle Tracking Analysis (NTA) on a Nanosight NS300 machine (Malvern Panalytical), the increase in the concentration of exosomes was observed for both animals; as shown in FIG. 7, the FUS-BBBO only macaque had an increase of approximately 54% and the FUS-BBBO, ThUS-BBBO and AAV macaque exhibited an increase of 34%.

Exosomes in the blood of Alzheimer's disease patients: during the phase 1 clinical with Alzheimer's disease (AD) patients (NCT04118764), we collected blood samples at baseline before FUS-BBBO, 1 hour, and 3 days after FUS-BBBO. Serum was extracted by centrifugation, and exosomes were isolated using ExoQuick. Nanoparticle tracking analysis (NTA) was carried out on a Nanosight N300 (Malvern Panalytical) to quantify the concentration of exosomes. A significant increase was observed 1 hour after FUS-BBBO, with a near return to baseline levels at the 3-day timepoint. Additionally, the 3-day concentration of extracellular vesicles had a positive correlation with the BBB opening volume, as calculated using contrast-enhanced MRI.

Apart from NTA, the content of serum-derived EVs was analyzed for neurological biomarkers of AD. A Luminex multiplex assay was used to detect and quantify AD-related proteins such as amyloid beta and tau in both serum and isolated extracellular vesicles. As shown in FIGS. 8A-8B, while the concentration of most analyzed proteins remains mostly unchanged 1 hour after treatment compared to baseline, a significant increase in the concentration of biomarkers in EVs was detected at the 3-day time point. A corresponding change in free biomarker concentration in serum was not observed, showing the advantages of using EVs as biomarker carriers in liquid biopsy applications. The level of some AD biomarkers in EVs is positively correlated with the volume of BBB opening, showing an increase in EVs released from the brain. Overall, these results support using EV analysis to detect important neurological biomarkers in neurodegenerative diseases.

Unless otherwise defined, all technical and scientific terms used herein have the same meaning as commonly understood by one of ordinary skill in the art. In case of conflict, the present document, including definitions, will control. Certain methods and materials are described below, although methods and materials similar or equivalent to those described herein can be used in the practice or testing of the presently disclosed subject matter. All publications, patent applications, patents, and other references mentioned herein are incorporated by reference in their entirety. The materials, methods, and examples disclosed herein are illustrative only and not intended to be limiting.

While it will become apparent that the subject matter herein described is well calculated to achieve the benefits and advantages set forth above, the presently disclosed subject matter is not to be limited in scope by the specific embodiments described herein. It will be appreciated that the disclosed subject matter is susceptible to modification, variation, and change without departing from the spirit thereof. Those skilled in the art will recognize or be able to ascertain, using no more than routine experimentation, many equivalents to the specific embodiments described herein. Such equivalents are intended to be encompassed by the following claims.

What is claimed is:

1. A system for releasing extracellular vesicles (EVs) of a subject, comprising:
an ultrasound assembly, including an ultrasound transducer and a function generator, configured to apply an ultrasound treatment to a target tissue of the subject;
a sampling device configured to collect a sample from the subject; and
an analysis device configured to analyze a biomarker in the sample.

2. The system of claim 1, wherein the ultrasound transducer comprises a spherical-segment focused ultrasound transducer, a pulse-echo ultrasound transducer, a linear array transducer, or combinations thereof.

3. The system of claim 1, wherein the ultrasound assembly is configured to apply a focused ultrasound (FUS) treatment, a theranostic-ultrasound (ThUS) treatment, or a combination thereof.

4. The system of claim 1, wherein the ultrasound treatment has one or more ultrasound parameters, wherein the ultrasound parameters comprise a center frequency, a focal depth, a focal area, one of a peak negative pressure, a stimulation duration, a duty cycle, a pulse repetition frequency (PRF), or combinations thereof.

5. The system of claim 1, wherein the transducer is configured to apply the ultrasound treatment to a blood-brain barrier.

6. The system of claim 1, wherein the sample comprises blood, serum, or a combination thereof.

7. The system of claim 1, wherein the biomarker comprises an inflammation-related marker, a proliferation-associated gene, an immediate inflammatory response-related protein, a hemoglobin-associated protein, or combinations thereof.

8. The system of claim 1, wherein the biomarker comprises alpha-synuclein (aSyn), amyloid-beta, tau, or combinations thereof.

9. The system of claim 2, wherein the ultrasound transducer is configured to apply the FUS treatment at about 0.25 MHz center frequency or the ThUS treatment at about 0.5 MHz center frequency.

10. The system of claim 1, wherein the sample is collected about 1 hour after the ultrasound treatment.

11. A method for releasing extracellular vesicles (EVs) of a subject, comprising:
applying an ultrasound treatment to a target tissue of the subject;
collecting a sample from the subject;
isolating EVs from the sample; and
analyzing a biomarker in the sample.

12. The method of claim 10, the ultrasound treatment comprises a focus ultrasound (FUS) treatment, a theranostic-ultrasound (ThUS) treatment, or a combination thereof.

13. The method of claim 10, wherein the sample comprises blood, serum, or a combination thereof.

14. The method of claim 10, wherein the target tissue comprises a brain and/or a blood-brain barrier.

15. The method of claim 10, wherein the biomarker comprises alpha-synuclein (aSyn), amyloid-beta, tau, or combinations thereof.

16. The method of claim 10, wherein the biomarker comprises an inflammation-related marker, a proliferation-associated gene, an immediate inflammatory response-related protein, a hemoglobin-associated protein, or combinations thereof.

17. The method of claim 10, wherein the sample is collected about 1 hour after the ultrasound treatment.

18. The method of claim 10, wherein the subject has a neurological disorder.

19. The method of claim 10, further comprising applying an effective amount of a therapeutic treatment to a subject based on the biomarker.

20. The method of claim 10, further comprising adjusting a parameter of the ultrasound treatment, wherein the parameter comprises a center frequency, a focal depth, a focal area, one of a peak negative pressure, a stimulation duration, a duty cycle, a pulse repetition frequency (PRF), or combinations thereof.
